(19) United States
(12) Patent Application Publication
(10) Pub. No.: US 2025/0166732 A1
(43) Pub. Date: May 22, 2025

(54) METAGENOMICS FOR MICROORGANISM IDENTIFICATION

(71) Applicant: Agency for Science, Technology and Research, Singapore (SG)

(72) Inventors: Niranjan Nagarajan, Singapore (SG); Chayaporn Suphavilai, Singapore (SG); Kwan Ki Ko, Singapore (SG); Kern Rei Chng, Singapore (SG); Kar Mun Lim, Singapore (SG)

(21) Appl. No.: 18/849,415

(22) PCT Filed: Mar. 9, 2023
(86) PCT No.: PCT/SG2023/050148

§ 371 Date: Sep. 20, 2024

(30) Foreign Application Priority Data
Mar. 23, 2022 (SG) 10202202957T

(51) Int. Cl. G16B 30/10; G16H 10/40; G16H 50/20

(52) U.S. Cl. CPC G16B 30/10; G16H 10/40; G16H 50/20

(57) ABSTRACT

Clinical decision support systems and methods for microorganism identification in a sample by determining long-read nucleic acid fragment sequence data (LRS data) originating from a plurality of species in the sample; performing taxonomic classification of the LRS data; determining an abundance levels of a plurality of reference genomes based on taxonomic identifiers of the LRS data; aligning the LRS data with a subset of the reference genomes; performing coverage analysis to determine an identity of one or more microorganism present in the sample based on the coverage analysis.

### TECHNICAL FIELD

This disclosure generally relates to systems and methods for microorganism identification based on metagenomic data.

### BACKGROUND

This background description is provided to generally present the context of the disclosure. Contents of this background section are neither expressly nor impliedly admitted as prior art against the present disclosure.

Current clinical diagnosis of infectious diseases relies on the identification of causative microorganisms in the laboratory. Accurate and rapid identification of microorganisms is essential for antimicrobial therapeutic optimization and rationalization. Gold standard laboratory identification of microorganisms often requires the culture of microorganisms. Culture-based methods are growth-dependent and have inherent biases for non-fastidious, rapid-growing species. Due to the growth-dependent nature of culture-based methods, the time taken to determine the presence of a microorganism in a species may vary from days to weeks, depending on the growth rate of the microorganism. Microbial pathogens that are non-viable or non-culturable in the media used are missed by culture-based methods.

Molecular diagnostics, such as targeted polymerase chain reaction (PCR) assays, are increasingly used in clinical settings to shorten the time taken to clinically actionable results. However, simple targeted PCR assays require a priori knowledge of the potential pathogens, and all non-targeted pathogens and intended pathogens with mutation(s) in the targeted (primed) sites are missed. Furthermore, it is extremely challenging to design PCR primers with both high specificity and sensitivity to a particular pathogen strain.

It is desired to address or ameliorate one or more disadvantages or limitations associated with the prior art, or to at least provide a useful alternative.

### SUMMARY

Some embodiments relate to a clinical decision support system comprising one or more processing units configured to:
receive long-read nucleic acid sequence data (LRS data) obtained from a sample, the LRS data comprising a plurality of records;
perform taxonomic classification of the LRS data to assign one or more taxonomic identifiers to each record on the LRS data;
determine abundance levels of a plurality of reference genomes in the sample based on the taxonomic identifiers;
align the LRS data with a subset of the reference genomes, wherein the subset of reference genomes demonstrated abundance in the sample reaching or exceeding a predefined abundance level;
perform coverage analysis based on the alignment of the LRS data with the subset of reference genomes to obtain a coverage estimate of each of the subset of reference genomes; and
identify one or more microorganism species present in the sample based on the coverage estimate.

In some embodiments, the LRS data is obtained from culture-free clinical samples.

In some embodiments, each record of the LRS data comprises data of at least 1,000 base pairs.

In some embodiments, the determination of the LRS data is performed in parallel with taxonomic classification.

In some embodiments, the determination of the LRS data, taxonomic classification and coverage analysis steps are performed in parallel.

In some embodiments, the coverage analysis is performed using a statistical distribution to estimate a breadth of coverage of the subset of reference genomes by the LRS data; optionally wherein the statistical distribution is a Poisson distribution or a negative binomial distribution.

In some embodiments, the at least one processing unit is further configured to align records in the LRS data with records in an antimicrobial resistance genome database to determine presence of antimicrobial resistant species in the sample.

In some embodiments, performing taxonomic classification comprises determining a K-mer profile of each record in the LRS data.

In some embodiments, the K value is in the range of 3 to 31 nucleotides.

In some embodiments, assigning one or more taxonomic identifiers to each record in the LRS data is based on the K-mer profile of the respective records.

In some embodiments, the taxonomic identifiers represent an operational taxonomic unit (OTU) referring to one or a combination of one or more of: domain, kingdom, phylum, class, order, family, genus, species, strain, or individual genome.

In some embodiments, the subset of genomes are selected based on the identified OTU.

In some embodiments, the aligning the LRS data to the subset of reference genomes is based on a total number of matched nucleotides and a read coverage score.

In some embodiments, coverage analysis comprises determining a percentage of breadth of coverage of each genome in the subset of reference genome by the LRS data.

Some embodiments relate to a computer-implemented method for microorganism identification, the method comprising:
receiving long-read nucleic acid fragment sequence data (LRS data) obtained from the sample;
performing taxonomic classification of the LRS data to assign one or more taxonomic identifiers to each record on the LRS data;
determining an abundance levels of a plurality of reference genomes based on the taxonomic identifiers of the LRS data;
aligning the LRS data with the subset of the reference genomes, wherein the subset of reference genomes demonstrated abundance in the sample reaching or exceeding a predefined abundance level;
performing coverage analysis based on the alignment of the LRS data with the candidate genomes;
identifying one or more microorganism species present in the sample based on the coverage estimate.

Some embodiments relate to a method for detecting infection by one or more microorganism in a subject, the method comprising:
determining long-read nucleic acid fragment sequence data (LRS data) from a sample obtained from the subject;
performing taxonomic classification of the LRS data to assign one or more taxonomic identifiers to each record on the LRS data;
determining abundance levels of a plurality of reference genomes based on the taxonomic identifiers of the LRS data;
aligning the LRS data with the candidate genomes in response to one or more candidate genomes of the plurality of reference genomes reaching or exceeding a predefined abundance level;
performing coverage analysis based on the alignment of the LRS data with the candidate genomes;
determining an identity of one or more microorganism present in the sample based on the coverage analysis so as to detect infection by the one or more microorganism in the subject.

### BRIEF DESCRIPTION OF THE DRAWINGS

Exemplary embodiments of the present invention are illustrated by way of example in the accompanying drawings in which like reference numbers indicate the same or similar elements and in which:

FIG. 1 is a schematic diagram illustrating a part of a method according to the disclosure;

FIG. 2 is another schematic illustrating a part of a method according to the disclosure; and

FIG. 3 is a block diagram of a system according to the disclosure.

### DETAILED DESCRIPTION

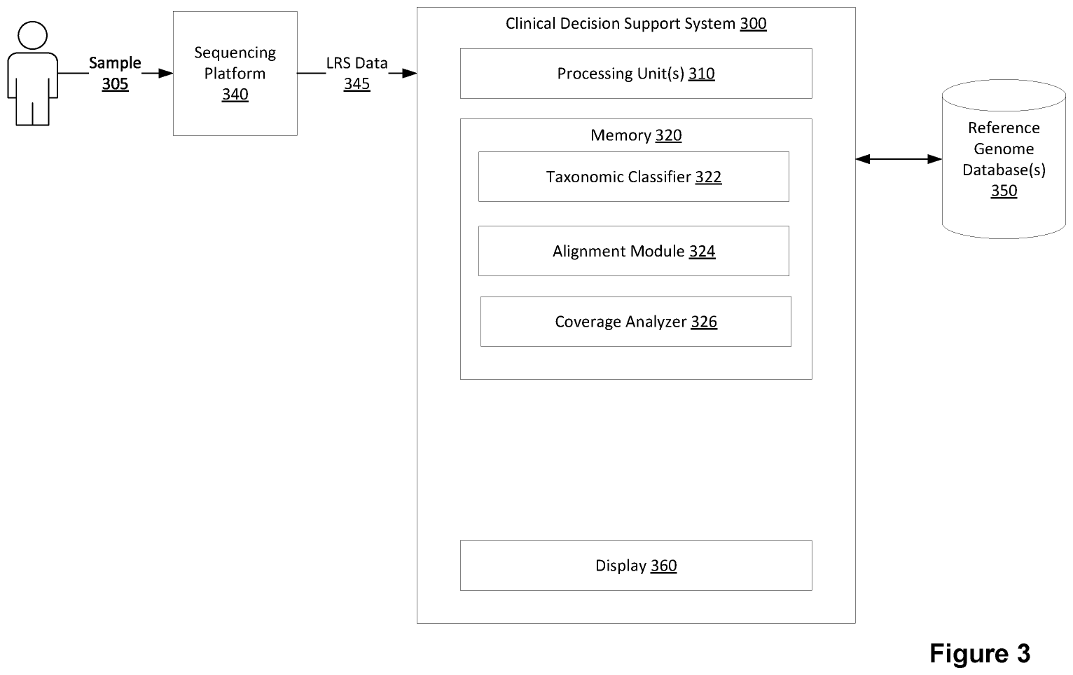

The disclosure related to systems for identifying pathogens in samples obtained from humans or other animals. The embodiments identify pathogens using genetic and metagenomic sequence-based technology that is accurate, fast and unbiased. The embodiments provide culture-free identification of unknown pathogens to improve the speed and accuracy of detection of pathogens in samples and shorten the time to generate information to drive efficacious therapy. Some embodiments relate to clinical decision support systems (300 of FIG. 3) that generate information relating to identity of pathogens present in a samples based on sequencing data originating from the sample data. The decision support systems aid clinical decision making including decisions relating to treatment based on the identity of the pathogen. The clinical decision support system of some embodiments may also generate a report including details of the identity of pathogens identified, coverage analysis statistics etc. The embodiments may be deployed in clinical settings such as hospitals to provide all-in-one microbial intelligence service. Some embodiments also detect anti-microbial resistant (AMR) strains of pathogens in samples.

The embodiments streamline laboratory processing protocols and advanced computational algorithms for metagenomic pathogen detection and identification in clinical samples. The embodiments can be applied directly on culture-free clinical samples such as sputum, bronchoalveolar lavage (BAL), swabs and blood culture samples to detect and identify microbial species present in the samples.

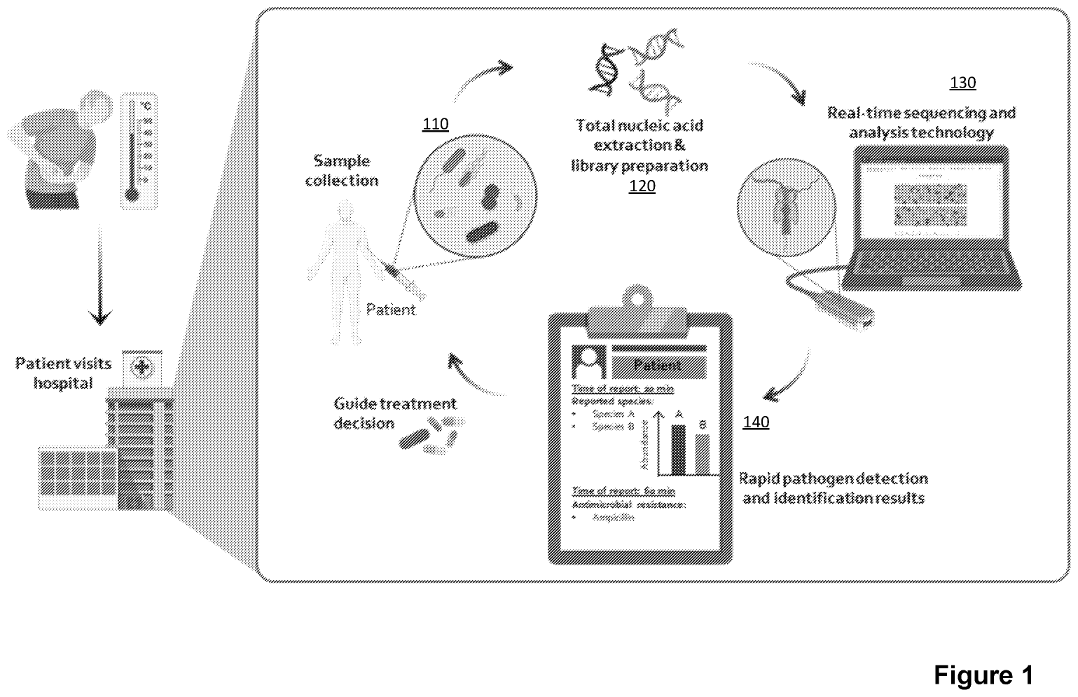

FIG. 1 illustrates a schematic diagram of a part of the technology that enables the identification of microbial species present in clinical samples (110) by metagenomic sequencing. The real-time, unbiased sequencing by the embodiments allows all or most clinically relevant pathogens present in a sample to be detected within an actionable time frame. An aliquot of the clinical sample, which may contain viral, bacterial or fungal pathogen(s), is subjected to lysis and total nucleic acid extraction (step 120). The total nucleic acid extract is then used for library preparation for downstream nanopore long-read DNA sequencing.

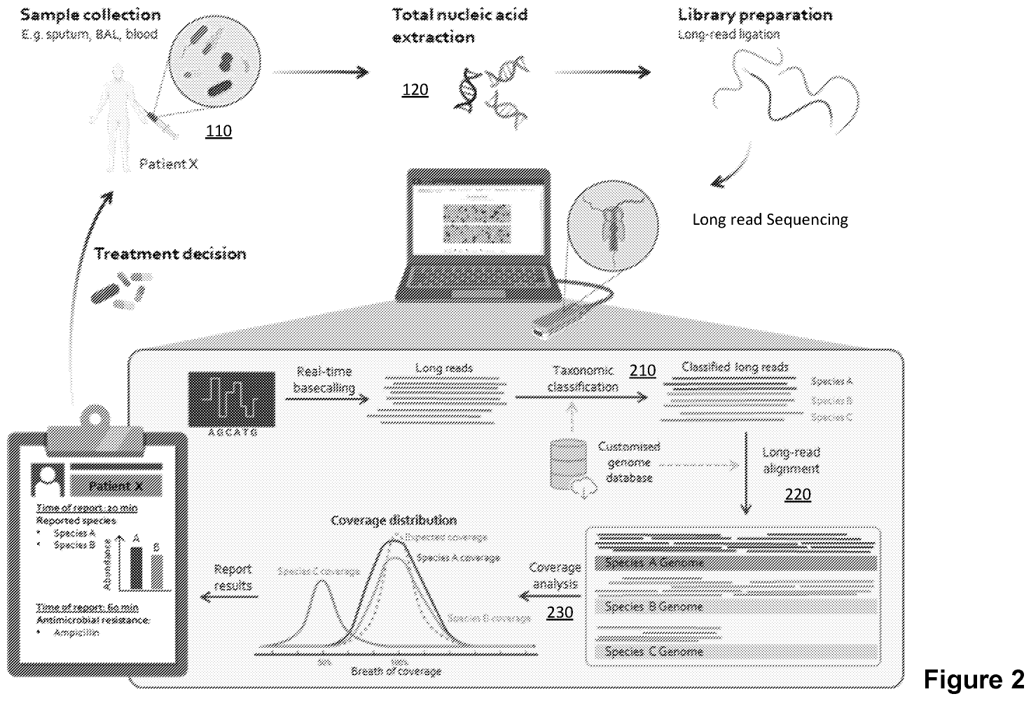

The real-time analysis algorithm of the embodiment is initiated once sequencing begins. DNA sequences are processed by the algorithm in real-time, and the platform reports results to the users once a microbial species is detected with a high confidence. The sequence-based technology of the embodiments is developed for direct pathogen detection and identification from clinical samples. The technology of the embodiments may be integrated with laboratory protocols and the computational algorithms of the embodiments that process DNA sequences data in real-time. FIG. 2 illustrates several components of the embodiments performing pathogen detection and identification.

FIG. 3 illustrates a clinical decision support system 300 and its associated components including a sequencing platform 340 and a reference genome database 350. A biological sample 305 is obtained from a person. The sample is processed by a sequencing platform 340 that generates long-read sequencing data (LRS Data 345). The LRS data is processed by the decision support system 300 to identify one or more microorganism species present in the sample. The decision support system comprises at least one processing unit 310 and a memory 320 comprising instructions to implement the various data processing algorithms/modules of the embodiments. The modules include a taxonomic classifier 322, alignment module 324 and a coverage analyzer 326. The clinical decision support system 300 also comprises a display 360 for presenting the results generated by the decision support system.

This disclosure contemplates any suitable number of systems 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 300 may include one or more computer systems 300; be unitary or distributed; span multiple locations; span multiple machines; span multiple data centers; or reside in a cloud, which may include one or more cloud components in one or more networks. Where appropriate, one or more computer systems 300 may perform without substantial spatial or temporal limitation one or more steps of one or more methods described or illustrated herein. One or more computer systems 300 may perform at different times or at different locations one or more steps of one or more methods described or illustrated herein, where appropriate.

### Clinical Sample Processing, Nucleic Acid Extraction and Library Preparation

Routine clinical samples, such as bronchoalveolar lavage (BAL), screening swabs and blood culture samples, are collected from patients as per routine clinical practice. The samples may be collected aseptically in sterile containers and transported to the onsite hospital diagnostic laboratory for processing within 1 hour of collection. In some embodiments, total nucleic acid extraction and library preparation may be performed as per nanopore long-read sequencing protocols (e.g. SQK-LSK109/LSK110). Embodiments may incorporate alternative sequencing technologies suited for the purpose of pathogen identification. When MinION flow cells are used, a maximum of 24 or 96 samples (depending on the choice of barcoding kits) can be sequenced in the same run. The analysis technology of the embodiments is applicable for any long-read DNA sequencing platforms, which can produce “reads” with at least 1,000 bases in length. Some embodiments may incorporate the nanopore sequencing platform to obtain the long-read DNA sequencing data. The sequencing data may be referred to as long-read nucleic acid sequence data (LRS data). The LRS data comprises a plurality of records, wherein each record relates to a specific sequencing read obtained from the sample.

### Pathogen Identification and Detection

Once the sequencing begins, each read as it is received by the system 300 is classified to a species by using the rapid taxonomic classifier 322 based on the curated genome database 350 (step 210 of FIG. 2). The pathogen identification process is performed continuously as the LRS data is received by the system 300. The system 300 keeps track of the abundance of the identified species in a sample as the LRS data is progressively received. Depending on the sequencing throughput, once the species abundance reaches a certain threshold (i.e. the total number of bases is equal to or greater than the genome size of a given species), the algorithm selects representative genomes associated with the specie. DNA sequences (or reads) are aligned to the representative genomes using a long-read alignment tool (alignment module 324, step 220 of FIG. 2) as illustrated in the schematic diagram of FIG. 2.

The sequencing of long-read DNA sequencing fragment data is performed over several intervals. For each interval, the embodiments obtain DNA sequence data that may be stored in a FASTQ format file, which contains multiple “reads”, i.e., DNA fragments with different lengths (1000-100,000 nucleotides). K-mer profile is extracted for each read. K-mer refers to all subsequences of a read with length K, where K ranges from 3 to 31 nucleotides.

The taxonomic classifier assigns one or more taxonomic identifiers to each read based on the K-mer profile and the reference genome database 350 accessible to the taxonomic classifier. The taxonomic identifier represents an operational taxonomic unit (OTU). The OTU might refer to domain, kingdom, phylum, class, order, family, genus, species, strain, or individual genome. The reference genome database may comprise DNA sequences of microbial genomes that are intended to be detected in the sample. The breadth of the identification capability of the system can be advantageously extended by expanding the reference genome database to cover a larger number of species. As more LRS data is received from the sequencing platform, the system 300 incrementally updates the count for each OTU (for example species-level OTU count). As the abundance level of a subset of OTUs reaches or exceeds a predefined threshold, such OTUs are earmarked as a subset of reference genomes to focus the subsequent analysis by the convergence analyzer. The system 300 monitors the OTU count and starts coverage analysis for subset of reference genomes when the corresponding species count/OTU count passes a threshold. The threshold may be defined based on the total number of sequenced nucleotides and the genome size of each species.

Coverage analysis (step 230 of FIG. 2) is performed by comparing the observed and the expected breadth of coverage of the LRS data in relation to the associated reference genome to detect the presence of the species. Based on the assumption that a whole genome is being sequenced, a Poisson distribution may be used for estimating the breadth of coverage given the number of total sequenced bases. Other alternative distributions modelling sequencing coverage may alternatively be incorporated. The alternative distributions include a negative binomial distribution. This step advantageously reduces the false positive rate that is caused by nanopore sequencing error or noise in the genome database. This reduction in false-positive results enables the algorithm of the embodiments to outperform existing algorithms (see performance comparison table below).

The embodiments may select a subset of reference genomes for each species based on the OTU count at strain or individual genome level. The embodiments align the classified reads to the associated reference genome and may identify genomic regions that are covered by the reads. A read may be said to align with a genome if an identity score (total number of matched nucleotides/alignment length×100) is at least 80% or 85% or 90% and/or a read-coverage score (alignment length/read length×100) is at least 80% or 85% or 90%. Given the alignment records, the embodiments calculate the percentage of the breadth of coverage for each species. The embodiment may report the presence of the species when the coverage percentage is at least 40-90% of the expected coverage of each species. The expected coverage percentage may follow a Poisson distribution, which takes into account the total number of sequenced nucleotides and the genome size of each species. In parallel with the pathogen detection and identification module, an additional antimicrobial resistance (AMR) module may align each read to an AMR gene records in the reference genome database. The AMR gene records contain DNA sequences of genes that have previously been reported as indicators of antimicrobial resistance. An LRS read may be said to align with an AMR gene if an identity score is at least 80%, 85% or 90% and a gene-coverage score (alignment length/gene length×100) is at least 80%, 85% or 90%. The system reports a list of AMR genes detected within the input sample.

### Performance Comparison

To compare the detection and identification performance of the embodiments, nanopore long-read sequencing data of 41 direct clinical samples was obtained from 38 sputum, 2 endotracheal tube aspirate (ETA), and 1 bronchoalveolar lavage (BAL). The percentage of the human genome in the samples ranged from 0.14% to 83.71%. The comparison reported microbial species detected by culture-based and qPCR-based methods, as well as microbial species identified by their metagenomic pipeline.

The results of the metagenomic pipeline proposed in Charalampous, T., Kay, G. L., Richardson, H., Aydin, A., Baldan, R., Jeanes, C., . . . & O'Grady, J. (2019) Nanopore metagenomics enables rapid clinical diagnosis of bacterial lower respiratory infection, Nature Biotechnology, 37(7), 783-792 was compared with results obtained using the embodiments, using culture and qPCR results as ground truth as illustrated in Table 1 below. Across 41 samples, the presence of 44 pathogens was confirmed by either culture or qPCR methods, and 8 pathogens were confirmed to be negative. Charalampous et al. could identify all 44 pathogens (100% sensitivity), while the embodiments according to the disclosure identified 39 pathogens (89% sensitivity). However, Charalampous et al. identified 6 species that were confirmed to be negative by qPCR (25% specificity), while embodiments according to the disclosure reported none of such erroneously identified species (100% specificity). Additionally, 9 pathogens were not identified by culture methods, but were identified by metagenomic methods and confirmed by qPCR. These results demonstrate the advantage of embodiments according to the disclosure, where pathogens undetected by the prior art methods are readily detected by metagenomic sequencing.

TABLE 1
Comparison of microbial species detected by culturing-based and qPCR-based methods, Charalampous et al. (2019), and the disclosed embodiment

| | | | | | | | Organism identified | | | |
|---|---|---|---|---|---|---|---|---|---|---|
| | | | | # non- | | Confirmed | by metagenomic | Reported | | |
| | | % human | # | human | Confirmed positive | negative | pipeline in | species by the | | Reported AMR gene family by the |
| Sample | Sample type | reads | reads | reads | Specie | Species | Charalampous et al. | disclosed embodiments | Comments on results | disclosed embodiments |
| S1 | ETA | 0.24% | 108610 | 108346 | E. coli | | Escherichia coli | E. coli | | ANT(3″) |
| | | | | | | | | | | ATP-binding cassette (ABC) |
| | | | | | | | | | | antibiotic efflux pump |
| | | | | | | | | | | ATP-binding cassette (ABC) |
| | | | | | | | | | | antibiotic efflux pump; major |
| | | | | | | | | | | facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | General Bacterial Porin with |
| | | | | | | | | | | reduced permeability to beta- |
| | | | | | | | | | | lactams; resistance-nodulation-cell |
| | | | | | | | | | | division (RND) antibiotic efflux |
| | | | | | | | | | | pump |
| | | | | | | | | | | TEM beta-lactamase |
| | | | | | | | | | | ampC-type beta-lactamase |
| | | | | | | | | | | kdpDE |
| | | | | | | | | | | macrolide phosphotransferase |
| | | | | | | | | | | (MPH) |
| | | | | | | | | | | major facilitator superfamily (MFS) |
| | | | | | | | | | | antibiotic efflux pump |
| | | | | | | | | | | major facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | sulfonamide resistant sul |
| | | | | | | | | | | trimethoprim resistant dihydrofolate |
| | | | | | | | | | | reductase dfr |
| | | | | | | | | | | undecaprenyl pyrophosphate |
| | | | | | | | | | | related proteins |
| S2 | Sputum | 0.18% | 17516 | 17485 | K. pneumoniae | | Klebsiella | K. | | ATP-binding cassette (ABC) |
| | | | | | | | pneumoniae | pneumoniae | | antibiotic efflux pump |
| | | | | | | | Streptococcus | | | Erm 23S ribosomal RNA |
| | | | | | | | parasanguinis | | | methyltransferase |
| | | | | | | | | | | General Bacterial Porin with |
| | | | | | | | | | | reduced permeability to beta- |
| | | | | | | | | | | lactams |
| | | | | | | | | | | SHV beta-lactamase |
| | | | | | | | | | | fosfomycin thiol transferase |
| | | | | | | | | | | lincosamide nucleotidyltransferase |
| | | | | | | | | | | (LNU) |
| | | | | | | | | | | major facilitator superfamily (MFS) |
| | | | | | | | | | | antibiotic efflux pump |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| S3 | Sputum | 1.44% | 26641 | 26257 | P. aeruginosa | | Pseudomonas | P. aeruginosa | | OXA beta-lactamase |
| | | | | | | | aeruginosa | S. parasanguinis | | Outer Membrane Porin |
| | | | | | | | Streptococcus | | | (Opr); resistance-nodulation-cell |
| | | | | | | | oralis | | | division (RND) antibiotic efflux |
| | | | | | | | Streptococcus | | | pump |
| | | | | | | | parasanguinis | | | ciprofloxacin phosphotransferase |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | small multidrug resistance (SMR) |
| | | | | | | | | | | antibiotic efflux pump |
| S4 | Sputum | 0.14% | 7358 | 7348 | S. marcescens | | Serratia | S. marcescens | | |
| | | | | | | | marcescens | | | |
| | | | | | | | Pantoea | | | |
| | | | | | | | stewartii | | | |
| | | | | | | | Citrobacter | | | |
| | | | | | | | freundii | | | |
| | | | | | | | Streptococcus | | | |
| | | | | | | | parasanguinis | | | |
| | | | | | | | Salmonella | | | |
| | | | | | | | enterica | | | |
| | | | | | | | Serratia | | | |
| | | | | | | | plymuthica | | | |
| S5 | Sputum | 0.12% | 19888 | 19865 | K. oxytoca | K. pneumoniae | Klebsiella | K. oxytoca | Charalam | OXY beta-lactamase |
| | | | | | | | oxytoca | C. freundii | pous et | |
| | | | | | | | Citrobacter | K. sp. M5al | al. | |
| | | | | | | | freundii | | reported | |
| | | | | | | | Klebsiella sp. | | K. | |
| | | | | | | | M5al | | pneumoniae, | |
| | | | | | | | Klebsiella | | which | |
| | | | | | | | pneumoniae | | has been | |
| | | | | | | | Klebsiella | | confirmed | |
| | | | | | | | michiganensis | | to be | |
| | | | | | | | | | negative | |
| | | | | | | | | | by qPCR. | |
| S6 | Sputum | 18.95% | 16403 | 13295 | S. aureus | | Staphylococcus | S. aureus | | ATP-binding cassette (ABC) |
| | | | | | | | aureus | | | antibiotic efflux pump; major |
| | | | | | | | | | | facilitator superfamily (MFS) |
| | | | | | | | | | | antibiotic efflux pump |
| | | | | | | | | | | blaZ beta-lactamase |
| | | | | | | | | | | major facilitator superfamily (MFS) |
| | | | | | | | | | | antibiotic efflux pump |
| | | | | | | | | | | multidrug and toxic compound |
| | | | | | | | | | | extrusion (MATE) transporter |
| S7 | Sputum | 33.73% | 32730 | 21690 | H. influenzae | | Streptococcus | P. aeruginosa | Disclosed | CfxA beta-lactamase |
| | | | | | P. aeruginosa | | parasanguinis | S. parasanguinis | embodiments | resistance-nodulation-cell division |
| | | | | | | | Pseudomonas | S. sanguinis | missed | (RND) antibiotic efflux pump |
| | | | | | | | aeruginosa | V. parvula | H. influenzae | |
| | | | | | | | Veillonella | | | |
| | | | | | | | parvula | | | |
| | | | | | | | Rothia | | | |
| | | | | | | | mucilaginosa | | | |
| | | | | | | | Streptococcus | | | |
| | | | | | | | sanguinis | | | |
| | | | | | | | Haemophilus | | | |
| | | | | | | | influenzae | | | |
| | | | | | | | Haemophilus | | | |
| | | | | | | | parainfluenzae | | | |
| | | | | | | | Neisseria sicca | | | |
| S8 | Sputum | 10.47% | 49277 | 44120 | M. catarrhalis | S. pneumoniae | Moraxella | M. catarrhalis | Charalam | BRO Beta-lactamase |
| | | | | | | | catarrhalis | S. gordonii | pous et | intrinsic colistin resistant |
| | | | | | | | Streptococcus | S. parasanguinis | al. | phosphoethanolamine transferase |
| | | | | | | | parasanguinis | S. salivarius | reported | tetracycline-resistant ribosomal |
| | | | | | | | Streptococcus | V. parvula | S. pneumoniae, | protection protein |
| | | | | | | | mitis | R. mucilaginosa | which | |
| | | | | | | | Veillonella | | has been | |
| | | | | | | | parvula | | confirmed | |
| | | | | | | | Streptococcus | | to be | |
| | | | | | | | salivarius | | negative | |
| | | | | | | | Rothia | | by qPCR. | |
| | | | | | | | mucilaginosa | | | |
| | | | | | | | Streptococcus | | | |
| | | | | | | | pseudopneumoniae | | | |
| | | | | | | | Streptococcus | | | |
| | | | | | | | oralis | | | |
| | | | | | | | Streptococcus | | | |
| | | | | | | | pneumoniae | | | |
| | | | | | | | Neisseria sicca | | | |
| | | | | | | | Streptococcus | | | |
| | | | | | | | gordonii | | | |
| S9 | Sputum | 0.49% | 29111 | 28969 | E. coli | P aeruginosa* | Escherichia coli | E. coli | | AAC(3) |
| | | | | | | | Lactobacillus | L. paracasei | | ANT(3″) |
| | | | | | | | paracasei | L. casei | | ATP-binding cassette (ABC) |
| | | | | | | | Lactobacillus | | | antibiotic efflux pump |
| | | | | | | | casei | | | ATP-binding cassette (ABC) |
| | | | | | | | Candida | | | antibiotic efflux pump; major |
| | | | | | | | albicans | | | facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | Erm 23S ribosomal RNA |
| | | | | | | | | | | methyltransferase |
| | | | | | | | | | | TEM beta-lactamase |
| | | | | | | | | | | ampC-type beta-lactamase |
| | | | | | | | | | | macrolide phosphotransferase |
| | | | | | | | | | | (MPH) |
| | | | | | | | | | | major facilitator superfamily (MFS) |
| | | | | | | | | | | antibiotic efflux pump |
| | | | | | | | | | | major facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | trimethoprim resistant dihydrofolate |
| | | | | | | | | | | reductase dfr |
| | | | | | | | | | | undecaprenyl pyrophosphate |
| | | | | | | | | | | related proteins |
| S10 | Sputum | 7.85% | 56005 | 51610 | H. influenzae | | Haemophilus | H. influenzae | Disclosed | Erm 23S ribosomal RNA |
| | | | | | S. pneumoniae | | influenzae | S. pseudopneumoniae | embodiments | methyltransferase |
| | | | | | | | Streptococcus | S. sp. oral | missed S. | major facilitator superfamily (MFS) |
| | | | | | | | pseudopneumoniae | taxon 431 | pneumoniae | antibiotic efflux pump |
| | | | | | | | Streptococcus | S. parasanguinis | but | tetracycline-resistant ribosomal |
| | | | | | | | sp. oral taxon | H. parainfluenzae | reported | protection protein |
| | | | | | | | 431 | | S. pseudopneumoniae | |
| | | | | | | | Streptococcus | | instead | |
| | | | | | | | pneumoniae | | based on | |
| | | | | | | | Streptococcus | | coverage | |
| | | | | | | | parasanguinis | | analysis | |
| | | | | | | | Streptococcus | | | |
| | | | | | | | mitis | | | |
| S11 | Sputum | 4.98% | 43088 | 40944 | S. pneumoniae | | Streptococcus | N. subflava | Disclosed | TEM beta-lactamase |
| | | | | | | | parasanguinis | S. sp. A12 | embodiments | |
| | | | | | | | Streptococcus | H. parainfluenzae | missed | |
| | | | | | | | sp. A12 | S. australis | S. pneumoniae | |
| | | | | | | | Streptococcus | S. parasanguinis | | |
| | | | | | | | mitis | S. mitis | | |
| | | | | | | | Rothia | B. longum | | |
| | | | | | | | mucilaginosa | | | |
| | | | | | | | Bifidobacterium | | | |
| | | | | | | | longum | | | |
| | | | | | | | Streptococcus | | | |
| | | | | | | | pseudopneumoniae | | | |
| | | | | | | | Streptococcus | | | |
| | | | | | | | pneumoniae | | | |
| | | | | | | | Streptococcus | | | |
| | | | | | | | sp. I-P16 | | | |
| | | | | | | | Streptococcus | | | |
| | | | | | | | sp. I-G2 | | | |
| | | | | | | | Haemophilus | | | |
| | | | | | | | parainfluenzae | | | |
| S12 | Sputum | 11.54% | 43267 | 38274 | M. | H. | Haemophilus | M. catarrhalis | Charalam | BRO Beta-lactamase |
| | | | | | catarrhalis | influenzae | parainfluenzae | H. parainfluenzae | pous et al. | Erm 23S ribosomal RNA |
| | | | | | | | Moraxella | S. gordonii | reported | methyltransferase |
| | | | | | | | catarrhalis | S. parasanguinis | H. influenzae, | TEM beta-lactamase |
| | | | | | | | Streptococcus | | which | intrinsic colistin resistant |
| | | | | | | | gordonii | | has been | phosphoethanolamine transferase |
| | | | | | | | Neisseria sicca | | confirmed | |
| | | | | | | | Haemophilus | | to be | |
| | | | | | | | influenzae | | negative | |
| | | | | | | | Streptococcus | | by qPCR. | |
| | | | | | | | parasanguinis | | | |
| S13 | Sputum | 0.67% | 27592 | 27406 | S. marcescens | | Serratia | S. | | |
| | | | | | | | marcescens | marcescens | | |
| S14 | Sputum | 1.29% | 41154 | 40622 | S. aureus | | Staphylococcus | S. aureus | | ATP-binding cassette (ABC) |
| | | | | | M. catarrhalis | | aureus | M. catarrhalis | | antibiotic efflux pump; major |
| | | | | | | | Moraxella | G. | | facilitator superfamily (MFS) |
| | | | | | | | catarrhalis | morbillorum | | antibiotic efflux pump |
| | | | | | | | Rothia | R. | | BRO Beta-lactamase |
| | | | | | | | mucilaginosa | mucilaginosa | | blaZ beta-lactamase |
| | | | | | | | Streptococcus | S. | | intrinsic colistin resistant |
| | | | | | | | constellatus | constellatus | | phosphoethanolamine transferase |
| | | | | | | | Fusobacterium | S. | | major facilitator superfamily (MFS) |
| | | | | | | | nucleatum | parasanguinis | | antibiotic efflux pump |
| | | | | | | | Fusobacterium | S. anginosus | | multidrug and toxic compound |
| | | | | | | | periodonticum | | | extrusion (MATE) transporter |
| | | | | | | | Streptococcus | | | |
| | | | | | | | parasanguinis | | | |
| | | | | | | | Streptococcus | | | |
| | | | | | | | anginosus | | | |
| | | | | | | | Streptococcus | | | |
| | | | | | | | intermedius | | | |
| S15 | Sputum | 2.57% | 37500 | 36537 | S. aureus | S. | Staphylococcus | S. aureus | Charalam | blaZ beta-lactamase |
| | | | | | | pneumoniae | aureus | S. oralis | pous et | major facilitator superfamily (MFS) |
| | | | | | | | Streptococcus | C. koseri | al. | antibiotic efflux pump |
| | | | | | | | mitis | R. | reported | multidrug and toxic compound |
| | | | | | | | Streptococcus | mucilaginosa | S. | extrusion (MATE) transporter |
| | | | | | | | oralis | H. | pneumoniae, | |
| | | | | | | | Streptococcus | parainfluenzae | which | |
| | | | | | | | parasanguinis | N. subflava | has been | |
| | | | | | | | Citrobacter | S. parasanguinis | confirmed | |
| | | | | | | | koseri | | to be | |
| | | | | | | | Rothia | | negative | |
| | | | | | | | mucilaginosa | | by qPCR. | |
| | | | | | | | Streptococcus | | | |
| | | | | | | | salivarius | | | |
| | | | | | | | Haemophilus | | | |
| | | | | | | | parainfluenzae | | | |
| | | | | | | | Streptococcus | | | |
| | | | | | | | pseudopneumoniae | | | |
| | | | | | | | Streptococcus | | | |
| | | | | | | | pneumoniae | | | |
| | | | | | | | Prevotella | | | |
| | | | | | | | melaninogenica | | | |
| S16 | Sputum | 0.28% | 85298 | 85057 | S. | | Staphylococcus | S. aureus | | ATP-binding cassette (ABC) |
| | | | | | aureus | | aureus | C. | | antibiotic efflux pump; major |
| | | | | | | | Streptococcus | kroppenstedtii | | facilitator superfamily (MFS) |
| | | | | | | | oralis | S. oralis | | antibiotic efflux pump |
| | | | | | | | Lactobacillus | L. rhamnosus | | Erm 23S ribosomal RNA |
| | | | | | | | rhamnosus | | | methyltransferase |
| | | | | | | | | | | Target protecting FusB-type protein |
| | | | | | | | | | | conferring resistance to Fusidic |
| | | | | | | | | | | acid |
| | | | | | | | | | | blaZ beta-lactamase |
| | | | | | | | | | | fosfomycin thiol transferase |
| | | | | | | | | | | major facilitator superfamily (MFS) |
| | | | | | | | | | | antibiotic efflux pump |
| | | | | | | | | | | methicillin resistant PBP2 |
| | | | | | | | | | | multidrug and toxic compound |
| | | | | | | | | | | extrusion (MATE) transporter |
| S17 | Sputum | 7.39% | 25499 | 23615 | | | Streptococcus | S. equinus | | CfxA beta-lactamase |
| | | | | | | | oralis | S. salivarius | | Erm 23S ribosomal RNA |
| | | | | | | | Streptococcus | S. sp. oral | | methyltransferase |
| | | | | | | | salivarius | taxon 431 | | TEM beta-lactamase |
| | | | | | | | Prevotella | S. oralis | | tetracycline-resistant ribosomal |
| | | | | | | | melaninogenica | S. sp. A12 | | protection protein |
| | | | | | | | Streptococcus | P. | | |
| | | | | | | | parasanguinis | melaninogenica | | |
| | | | | | | | Streptococcus | S. | | |
| | | | | | | | sp. oral taxon | parasanguinis | | |
| | | | | | | | 431 | | | |
| | | | | | | | Streptococcus | | | |
| | | | | | | | sp. A12 | | | |
| | | | | | | | Streptococcus | | | |
| | | | | | | | sp. NPS 308 | | | |
| | | | | | | | Streptococcus | | | |
| | | | | | | | sp. FDAARGOS_192 | | | |
| S18 | Sputum | 0.41% | 38902 | 38744 | H. | | Haemophilus | H. influenzae | | Intrinsic peptide antibiotic resistant |
| | | | | | influenzae | | influenzae | | | Lps |
| | | | | | | | | | | TEM beta-lactamase |
| | | | | | | | | | | multidrug and toxic compound |
| | | | | | | | | | | extrusion (MATE) transporter |
| S19 | Sputum | 11.63% | 47994 | 42413 | | | Streptococcus | R. | | APH(3′) |
| | | | | | | | salivarius | mucilaginosa | | Erm 23S ribosomal RNA |
| | | | | | | | Streptococcus | S. salivarius | | methyltransferase |
| | | | | | | | parasanguinis | S. equinus | | TEM beta-lactamase |
| | | | | | | | Streptococcus | S. | | tetracycline-resistant ribosomal |
| | | | | | | | mitis | parasanguinis | | protection protein |
| | | | | | | | Streptococcus | S. mitis | | |
| | | | | | | | sp. FDAARGOS_{—}192 | S. oralis | | |
| | | | | | | | Streptococcus | S. gordonii | | |
| | | | | | | | oralis | S. sanguinis | | |
| | | | | | | | Streptococcus | | | |
| | | | | | | | sanguinis | | | |
| | | | | | | | Rothia | | | |
| | | | | | | | mucilaginosa | | | |
| | | | | | | | Veillonella | | | |
| | | | | | | | parvula | | | |
| | | | | | | | Streptococcus | | | |
| | | | | | | | sp. oral taxon | | | |
| | | | | | | | 431 | | | |
| | | | | | | | Streptococcus | | | |
| | | | | | | | gordonii | | | |
| | | | | | | | Streptococcus | | | |
| | | | | | | | constellatus | | | |
| S20 | Sputum | 7.04% | 46331 | 43070 | H. | | Haemophilus | H. influenzae | | Intrinsic peptide antibiotic resistant |
| | | | | | influenzae | | influenzae | S. mitis | | Lps |
| | | | | | | | Rothia | S. | | tetracycline-resistant ribosomal |
| | | | | | | | mucilaginosa | parasanguinis | | protection protein |
| | | | | | | | Streptococcus | R. | | |
| | | | | | | | mitis | mucilaginosa | | |
| | | | | | | | Streptococcus | | | |
| | | | | | | | parasanguinis | | | |
| S21 | Sputum | 0.31% | 45214 | 45075 | H. | S. | Haemophilus | H. | Charalam | Intrinsic peptide antibiotic resistant |
| | | | | | influenzae | pneumoniae | influenzae | influenzae | pous et | Lps |
| | | | | | | | Streptococcus | S. salivarius | al. | tetracycline-resistant ribosomal |
| | | | | | | | mitis | S. | reported | protection protein |
| | | | | | | | Haemophilus | parasanguinis | S. | |
| | | | | | | | parainfluenzae | S. oralis | pneumoniae, | |
| | | | | | | | Streptococcus | S. mitis | which | |
| | | | | | | | parasanguinis | H. | has been | |
| | | | | | | | Streptococcus | parainfluenzae | confirmed | |
| | | | | | | | sp. oral taxon | | to be | |
| | | | | | | | 431 | | negative | |
| | | | | | | | Streptococcus | | by qPCR. | |
| | | | | | | | salivarius | | | |
| | | | | | | | Streptococcus | | | |
| | | | | | | | oralis | | | |
| | | | | | | | Streptococcus | | | |
| | | | | | | | pneumoniae | | | |
| S22 | Sputum | 1.79% | 36853 | 36194 | | | Streptococcus | S. sanguinis | | CfxA beta-lactamase |
| | | | | | | | parasanguinis | P. | | tetracycline-resistant ribosomal |
| | | | | | | | Prevotella | melaninogenica | | protection protein |
| | | | | | | | melaninogenica | S. cristatus | | |
| | | | | | | | Streptococcus | R. | | |
| | | | | | | | mitis | mucilaginosa | | |
| | | | | | | | Streptococcus | S. mitis | | |
| | | | | | | | sanguinis | S. | | |
| | | | | | | | Rothia | parasanguinis | | |
| | | | | | | | mucilaginosa | H. | | |
| | | | | | | | Veillonella | parainfluenzae | | |
| | | | | | | | parvula | | | |
| | | | | | | | Haemophilus | | | |
| | | | | | | | parainfluenzae | | | |
| | | | | | | | Streptococcus | | | |
| | | | | | | | salivarius | | | |
| | | | | | | | Streptococcus | | | |
| | | | | | | | sp. A12 | | | |
| | | | | | | | Streptococcus | | | |
| | | | | | | | cristatus | | | |
| S23 | Sputum | 0.62% | 33140 | 32933 | H. | | Haemophilus | H. influenzae | | Intrinsic peptide antibiotic resistant |
| | | | | | influenzae | | influenzae | S. | | Lps |
| | | | | | | | Streptococcus | pseudopneumoniae | | TEM beta-lactamase |
| | | | | | | | parasanguinis | S. | | major facilitator superfamily (MFS) |
| | | | | | | | Streptococcus | parasanguinis | | antibiotic efflux pump |
| | | | | | | | salivarius | V. parvula | | tetracycline-resistant ribosomal |
| | | | | | | | Streptococcus | S. oralis | | protection protein |
| | | | | | | | oralis | S. mitis | | |
| | | | | | | | Veillonella | | | |
| | | | | | | | parvula | | | |
| | | | | | | | Streptococcus | | | |
| | | | | | | | pseudopneumoniae | | | |
| | | | | | | | Streptococcus | | | |
| | | | | | | | mitis | | | |
| | | | | | | | Streptococcus | | | |
| | | | | | | | sp. FDAARGOS_192 | | | |
| S24 | Sputum | 1.84% | 58752 | 57669 | H. | | Haemophilus | H. influenzae | | Intrinsic peptide antibiotic resistant |
| | | | | | influenzae | | influenzae | S. oralis | | Lps |
| | | | | | | | Streptococcus | S. mitis | | major facilitator superfamily (MFS) |
| | | | | | | | parasanguinis | H. | | antibiotic efflux pump |
| | | | | | | | Haemophilus | parainfluenzae | | multidrug and toxic compound |
| | | | | | | | parainfluenzae | V. parvula | | extrusion (MATE) transporter |
| | | | | | | | Veillonella | S. | | tetracycline-resistant ribosomal |
| | | | | | | | parvula | parasanguinis | | protection protein |
| | | | | | | | Streptococcus | S. sanguinis | | |
| | | | | | | | oralis | S. salivarius | | |
| | | | | | | | Streptococcus | S. sp. oral | | |
| | | | | | | | salivarius | taxon 431 | | |
| | | | | | | | Streptococcus | | | |
| | | | | | | | sp. oral taxon | | | |
| | | | | | | | 431 | | | |
| S25 | Sputum | 2.22% | 36621 | 35808 | H. | | Haemophilus | H. influenzae | | Intrinsic peptide antibiotic resistant |
| | | | | | influenzae | | influenzae | | | Lps |
| S26 | Sputum | 3.22% | 38138 | 36910 | M. | | Streptococcus | M. catarrhalis | | intrinsic colistin resistant |
| | | | | | catarrhalis | | oralis | S. | | phosphoethanolamine transferase |
| | | | | | | | Streptococcus | thermophilus | | tetracycline-resistant ribosomal |
| | | | | | | | mutans | S. mutans | | protection protein |
| | | | | | | | Moraxella | V. parvula | | |
| | | | | | | | catarrhalis | S. oralis | | |
| | | | | | | | Veillonella | S. | | |
| | | | | | | | parvula | parasanguinis | | |
| | | | | | | | Streptococcus | R. | | |
| | | | | | | | parasanguinis | dentocariosa | | |
| | | | | | | | Streptococcus | S. salivarius | | |
| | | | | | | | salivarius | S. gordonii | | |
| | | | | | | | Streptococcus | | | |
| | | | | | | | gordonii | | | |
| | | | | | | | Rothia | | | |
| | | | | | | | dentocariosa | | | |
| | | | | | | | Lactobacillus | | | |
| | | | | | | | salivarius | | | |
| S27 | Sputum | 0.32% | 78311 | 78064 | H. | | Haemophilus | H. influenzae | S. | ATP-binding cassette (ABC) |
| | | | | | influenzae | | influenzae | S. aureus | pyogenes | antibiotic efflux pump; major |
| | | | | | S. | | Streptococcus | S. pyogenes | was not | facilitator superfamily (MFS) |
| | | | | | aureus | | pyogenes | S. | detectable | antibiotic efflux pump |
| | | | | | S. | | Staphylococcus | parasanguinis | by | Erm 23S ribosomal RNA |
| | | | | | pyogenes | | aureus | S. salivarius | culturing | methyltransferase |
| | | | | | | | Streptococcus | | but was | blaZ beta-lactamase |
| | | | | | | | salivarius | | confirmed | major facilitator superfamily (MFS) |
| | | | | | | | Streptococcus | | by qPCR | antibiotic efflux pump |
| | | | | | | | parasanguinis | | | multidrug and toxic compound |
| | | | | | | | | | | extrusion (MATE) transporter |
| S28 | Sputum | 3.09% | 35804 | 34699 | | S. pneumoniae | Streptococcus | R. mucilaginosa | Charalam | TEM beta-lactamase |
| | | | | | | | mitis | S. salivarius | pous et al. | |
| | | | | | | | Streptococcus | S. australis | reported | |
| | | | | | | | salivarius | V. parvula | S. pneumoniae, | |
| | | | | | | | Streptococcus | S. sanguinis | which has been | |
| | | | | | | | oralis | S. parasanguinis | confirmed | |
| | | | | | | | Veillonella | S. sp. A12 | to be | |
| | | | | | | | parvula | | negative | |
| | | | | | | | Streptococcus | | by qPCR. | |
| | | | | | | | sp. A12 | | | |
| | | | | | | | Streptococcus | | | |
| | | | | | | | sanguinis | | | |
| | | | | | | | Rothia | | | |
| | | | | | | | mucilaginosa | | | |
| | | | | | | | Streptococcus | | | |
| | | | | | | | sp. oral taxon | | | |
| | | | | | | | 431 | | | |
| | | | | | | | Streptococcus | | | |
| | | | | | | | parasanguinis | | | |
| | | | | | | | Streptococcus | | | |
| | | | | | | | pneumoniae | | | |
| | | | | | | | Streptococcus | | | |
| | | | | | | | pseudopneumoniae | | | |
| | | | | | | | Prevotella | | | |
| | | | | | | | melaninogenica | | | |
| | | | | | | | Streptococcus | | | |
| | | | | | | | sp. I-G2 | | | |
| | | | | | | | Streptococcus | | | |
| | | | | | | | sp. I-P16 | | | |
| | | | | | | | Haemophilus | | | |
| | | | | | | | parainfluenzae | | | |
| S29 | Sputum | 83.71% | 57865 | 9429 | P. | | Pseudomonas | P. aeruginosa | S. aureus | OXA beta-lactamase |
| | | | | | aeruginosa | | aeruginosa | S. aureus | was not | Outer Membrane Porin |
| | | | | | S. aureus | | Staphylococcus | | detectable | (Opr); resistance-nodulation-cell |
| | | | | | | | aureus | | by | division (RND) antibiotic efflux |
| | | | | | | | | | culturing | pump |
| | | | | | | | | | but was | TEM beta-lactamase |
| | | | | | | | | | confirmed | blaZ beta-lactamase |
| | | | | | | | | | by qPCR | chloramphenicol acetyltransferase |
| | | | | | | | | | | (CAT) |
| | | | | | | | | | | major facilitator superfamily (MFS) |
| | | | | | | | | | | antibiotic efflux pump |
| | | | | | | | | | | multidrug and toxic compound |
| | | | | | | | | | | extrusion (MATE) transporter |
| | | | | | | | | | | resistance-nodulation-cell division |
| | | | | | | | | | | (RND) antibiotic efflux pump |
| S30 | BAL | 0.56% | 27371 | 27217 | P. | | Pseudomonas | P. aeruginosa | | OXA beta-lactamase |
| | | | | | aeruginosa | | aeruginosa | | | Outer Membrane Porin |
| | | | | | | | | | | (Opr); resistance-nodulation-cell |
| | | | | | | | | | | division (RND) antibiotic efflux |
| | | | | | | | | | | pump |
| | | | | | | | | | | PDC beta-lactamase |
| | | | | | | | | | | TEM beta-lactamase |
| | | | | | | | | | | chloramphenicol acetyltransferase |
| | | | | | | | | | | (CAT) |
| | | | | | | | | | | ciprofloxacin phosphotransferase |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | small multidrug resistance (SMR) |
| | | | | | | | | | | antibiotic efflux pump |
| S31 | Sputum | 69.28% | 43823 | 13463 | H. influenzae | | Haemophilus | H. influenzae | | TEM beta-lactamase |
| | | | | | | | influenzae | S. anginosus | | |
| | | | | | | | Streptococcus | S. parasanguinis | | |
| | | | | | | | anginosus | | | |
| | | | | | | | Streptococcus | | | |
| | | | | | | | parasanguinis | | | |
| | | | | | | | Prevotella | | | |
| | | | | | | | intermedia | | | |
| | | | | | | | Tannerella | | | |
| | | | | | | | forsythia | | | |
| | | | | | | | Bifidobacterium | | | |
| | | | | | | | longum | | | |
| | | | | | | | Rothia | | | |
| | | | | | | | mucilaginosa | | | |
| | | | | | | | Streptococcus | | | |
| | | | | | | | gordonii | | | |
| | | | | | | | Veillonella | | | |
| | | | | | | | parvula | | | |
| | | | | | | | Streptococcus | | | |
| | | | | | | | oralis | | | |
| | | | | | | | Campylobacter | | | |
| | | | | | | | concisus | | | |
| S32 | Sputum | 0.59% | 48271 | 47988 | E. coli | | Escherichia coli | E. coli | | AAC(3) |
| | | | | | | | | | | ATP-binding cassette (ABC) |
| | | | | | | | | | | antibiotic efflux pump |
| | | | | | | | | | | ATP-binding cassette (ABC) |
| | | | | | | | | | | antibiotic efflux pump; major |
| | | | | | | | | | | facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | General Bacterial Porin with |
| | | | | | | | | | | reduced permeability to beta- |
| | | | | | | | | | | lactams; resistance-nodulation-cell |
| | | | | | | | | | | division (RND) antibiotic efflux |
| | | | | | | | | | | pump |
| | | | | | | | | | | TEM beta-lactamase |
| | | | | | | | | | | kdpDE |
| | | | | | | | | | | major facilitator superfamily (MFS) |
| | | | | | | | | | | antibiotic efflux pump |
| | | | | | | | | | | major facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | undecaprenyl pyrophosphate |
| | | | | | | | | | | related proteins |
| S33 | Sputum | 40.22% | 32085 | 19181 | | | Streptococcus | S. | | |
| | | | | | | | salivarius | salivarius | | |
| | | | | | | | Clavispora | S. | | |
| | | | | | | | lusitaniae | parasanguinis | | |
| | | | | | | | Streptococcus | S. equinus | | |
| | | | | | | | parasanguinis | | | |
| | | | | | | | Streptococcus | | | |
| | | | | | | | sp. FDAARGOS_192 | | | |
| | | | | | | | Rothia | | | |
| | | | | | | | mucilaginosa | | | |
| | | | | | | | Saccharomyces | | | |
| | | | | | | | cerevisiae | | | |
| S34 | Sputum | 13.36% | 40998 | 35522 | | | Candida | E. faecalis | | ATP-binding cassette (ABC) |
| | | | | | | | albicans | | | antibiotic efflux pump |
| | | | | | | | Enterococcus | | | Erm 23S ribosomal RNA |
| | | | | | | | faecalis | | | methyltransferase |
| | | | | | | | | | | TEM beta-lactamase |
| | | | | | | | | | | trimethoprim resistant dihydrofolate |
| | | | | | | | | | | reductase dfr |
| S35 | Sputum | 37.85% | 47893 | 29765 | E. coli | | Escherichia coli | E. coli | | ATP-binding cassette (ABC) |
| | | | | | | | | | | antibiotic efflux pump |
| | | | | | | | | | | ATP-binding cassette (ABC) |
| | | | | | | | | | | antibiotic efflux pump; major |
| | | | | | | | | | | facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | General Bacterial Porin with |
| | | | | | | | | | | reduced permeability to beta- |
| | | | | | | | | | | lactams; resistance-nodulation-cell |
| | | | | | | | | | | division (RND) antibiotic efflux |
| | | | | | | | | | | pump |
| | | | | | | | | | | TEM beta-lactamase |
| | | | | | | | | | | ampC-type beta-lactamase |
| | | | | | | | | | | kdpDE |
| | | | | | | | | | | major facilitator superfamily (MFS) |
| | | | | | | | | | | antibiotic efflux pump |
| | | | | | | | | | | major facilitator superfamily (MFS) |
| | | | | | | | | | | antibiotic efflux pump; resistance- |
| | | | | | | | | | | nodulation-cell division (RND) |
| | | | | | | | | | | antibiotic efflux pump |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | undecaprenyl pyrophosphate |
| | | | | | | | | | | related proteins |
| S36 | Sputum | 1.15% | 33155 | 32774 | H. | | Haemophilus | H. influenzae | | Erm 23S ribosomal RNA |
| | | | | | influenzae | | influenzae | V. parvula | | methyltransferase |
| | | | | | | | Veillonella | S. salivarius | | Intrinsic peptide antibiotic resistant |
| | | | | | | | parvula | S. mitis | | Lps |
| | | | | | | | Streptococcus | | | TEM beta-lactamase |
| | | | | | | | salivarius | | | multidrug and toxic compound |
| | | | | | | | Streptococcus | | | extrusion (MATE) transporter |
| | | | | | | | mitis | | | |
| S37 | Sputum | 0.68% | 60495 | 60083 | P. | | Pseudomonas | P. aeruginosa | | OXA beta-lactamase |
| | | | | | aeruginosa | | aeruginosa | | | Outer Membrane Porin |
| | | | | | | | | | | (Opr); resistance-nodulation-cell |
| | | | | | | | | | | division (RND) antibiotic efflux |
| | | | | | | | | | | pump |
| | | | | | | | | | | PDC beta-lactamase |
| | | | | | | | | | | TEM beta-lactamase |
| | | | | | | | | | | chloramphenicol acetyltransferase |
| | | | | | | | | | | (CAT) |
| | | | | | | | | | | ciprofloxacin phosphotransferase |
| | | | | | | | | | | pmr phosphoethanolamine |
| | | | | | | | | | | transferase |
| | | | | | | | | | | resistance-nodulation-cell division |
| | | | | | | | | | | (RND) antibiotic efflux pump |
| | | | | | | | | | | small multidrug resistance (SMR) |
| | | | | | | | | | | antibiotic efflux pump |
| S38 | Sputum | 45.80% | 23889 | 12947 | S. | | Staphylococcus | S. aureus | Disclopsed | ATP-binding cassette (ABC) |
| | | | | | aureus | | aureus | | embodiments | antibiotic efflux pump; major |
| | | | | | P. | | Pseudomonas | | missed P. | facilitator superfamily (MFS) |
| | | | | | aeruginosa | | aeruginosa | | aeruginosa | antibiotic efflux pump |
| | | | | | | | | | | TEM beta-lactamase |
| | | | | | | | | | | blaZ beta-lactamase |
| | | | | | | | | | | ciprofloxacin phosphotransferase |
| | | | | | | | | | | fosfomycin thiol transferase |
| | | | | | | | | | | major facilitator superfamily (MFS) |
| | | | | | | | | | | antibiotic efflux pump |
| | | | | | | | | | | multidrug and toxic compound |
| | | | | | | | | | | extrusion (MATE) transporter |
| | | | | | | | | | | resistance-nodulation-cell division |
| | | | | | | | | | | (RND) antibiotic efflux pump |
| S39 | Sputum | 0.57% | 60316 | 59972 | H. | | Moraxella | H. influenzae | M. | BRO Beta-lactamase |
| | | | | | influenzae | | catarrhalis | M. catarrhalis | catarrhalis | Erm 23S ribosomal RNA |
| | | | | | M. | | Haemophilus | S. | was not | methyltransferase |
| | | | | | catarrhalis | | influenzae | parasanguinis | detectable by | TEM beta-lactamase |
| | | | | | | | Streptococcus | S. anginosus | culturing | ciprofloxacin phosphotransferase |
| | | | | | | | parasanguinis | | but was | intrinsic colistin resistant |
| | | | | | | | | | confirmed | phosphoethanolamine transferase |
| | | | | | | | | | by qPCR | pmr phosphoethanolamine |
| | | | | | | | | | | transferase |
| S40 | ETA | 84.76% | 48848 | 7444 | S. | | Staphylococcus | S. | | ATP-binding cassette (ABC) |
| | | | | | aureus | | aureus | aureus | | antibiotic efflux pump; major |
| | | | | | | | Streptococcus | | | facilitator superfamily (MFS) |
| | | | | | | | constellatus | | | antibiotic efflux pump |
| | | | | | | | Streptococcus | | | TEM beta-lactamase |
| | | | | | | | anginosus | | | blaZ beta-lactamase |
| | | | | | | | Streptococcus | | | major facilitator superfamily (MFS) |
| | | | | | | | intermedius | | | antibiotic efflux pump |
| | | | | | | | | | | methicillin resistant PBP2 |
| | | | | | | | | | | multidrug and toxic compound |
| | | | | | | | | | | extrusion (MATE) transporter |
| S41 | Sputum | 8.23% | 2320 | 2129 | H. | E. coli | Staphylococcus | S. aureus | Charalam | ATP-binding cassette (ABC) |
| | | | | | influenzae | | aureus | | pous et | antibiotic efflux pump; major |
| | | | | | S. | | Haemophilus | | al. | facilitator superfamily (MFS) |
| | | | | | aureus | | influenzae | | reported | antibiotic efflux pump |
| | | | | | | | Neisseria sicca | | E. coli, | TEM beta-lactamase |
| | | | | | | | Escherichia coli | | which has | blaZ beta-lactamase |
| | | | | | | | | | been | fosfomycin thiol transferase |
| | | | | | | | | | confirmed | major facilitator superfamily (MFS) |
| | | | | | | | | | to be | antibiotic efflux pump |
| | | | | | | | | | negative | |
| | | | | | | | | | by qPCR. | |
| | | | | | | | | | Disclosed | |
| | | | | | | | | | embodiments | |
| | | | | | | | | | missed | |
| | | | | | | | | | H. influenzae | |

In summary, the technology according to the embodiments enables detection of species missed by routine clinical cultures that were confirmed by qPCR. Some embodiments also enable the detection of additional microbial species without the need for specific PCR primers. Some embodiments also advantageously improve specificity (100%) and overall accuracy (90%) compared to Charalampous et al in experiments undertaken to compare the performance of the embodiments as described above.

Integration with Clinical Practice

The technology of the embodiments utilizes nanopore long-read sequencing platforms which enable real-time analysis of DNA sequences as they become available. Once the sequencing starts, DNA reads are processed in real-time and an electronic or digital report documenting the findings is continuously updated as the sequencing progresses. The report may be presented on the display 360 of the system 300. When a new species (or new AMR genes) is detected and confirmed by coverage analysis, the report is updated accordingly. According to the results in Table 1, pathogens can be identified within 1-2 hours after sequencing initiation. Adding sample transport and processing (typically less than 2 hours), DNA extraction (typically less than 2 hours) and library preparation (about 4 hours) durations, the total turnaround time for identification of species in a sample could be less than 1 day. An electronic report documenting detected microbial species and AMR genes is generated within an actionable timeframe to guide clinical decision-making.

The technology of the embodiments can be deployed in a clinical laboratory. The streamlined laboratory protocols and algorithms can be used for detecting microbial species directly in clinical samples. The embodiments can be used in parallel or as a replacement of some of the conventional pathogen detection methods. The embodiments can also be used for challenging clinical cases where all routine pathogen detection tests are unyielding but clinical suspicion for infection remains. A list of exemplary hardware and software specifications used by some embodiments is provided in Table 2.

In some embodiments, an infection in a subject may be detected based on the identity of one or more microorganism present in the sample. The clinical decision support system may aid selection of a therapeutic agent to administer to the subject when infection by the one or more microorganism is detected in the subject.

TABLE 2
Exemplary hardware and software specifications

| Wet-lab related consumables | Wet-lab related equipment |
|---|---|
| Extraction kit e.g. Qiagen | Centrifuge with 50 mL tube |
| Powersoil Pro kit | holders and 2 mL tube holders |
| Beckman Coulter AMPure XP | Vortex with 2 mL tube adapters |
| Oxford Nanopore Technologies | Table-top centrifuge |
| Minion (FLO-MIN106D) or Flongle | DynaMag magnetic tube rack (for |
| (FLO-FLG001) flowcells | magnetic bead cleanups) |
| Oxford Nanopore Technologies | Oxford Nanopore Technologies |
| SQK-LSK109/LSK110 kit with | Minion Mk1b. If intend to use |
| NEBNext ® Companion Module. | Flongles flowcells, Flongle |
| If intend to multiplex, | adapter |
| NBD104 and NDB114, or | |
| NDB196 | |

Exemplary equipment and software for real-time analysis
A workstation computer or laptop
8-core CPU
16-32 GB RAM
1 TB SSD
Graphical Processing Unit (GPU) with at least 8 GB memory
Suitable software
MinKNOW for nanopore sequencing
Python 3.x for real-time analysis scripts
Guppy for live basecalling
Minimap2 for long-read alignment
Kraken2 for taxonomic classification

The embodiments provide algorithms for real-time analysis of long-read sequencing data generated from long-read DNA sequencing platforms. By utilizing the unique properties of long-read data, the algorithms identify microbial species within metagenomic samples and reduce the false-positive rate, improving overall accuracy over the existing metagenomic pipelines. The embodiments provide the ability to detect and identify pathogens and other microbial species in clinical samples directly, without the need for cultures or specific PCR. The embodiments require a smaller number of reads which can be obtained within 1-2 hours, shortening the time to detection which supports clinical decision-making in a timely manner. The technology setup for the embodiments is advantageously portable and can be deployed to any location with reliable electricity supplies.

In general, methods for identifying pathogens in metagenomic samples are designed for NGS technology (i.e. Illumina sequencing platform). The assumption that existing methods can be applied on sequencing data from any platform would lead to lower accuracy, as we observed in the performance comparison section. Our technology is designed for utilizing long-read information and supporting real-time analysis for long-read sequencing platforms.

The embodiments provide a flexible and scalable technology. Some embodiments allow processing a single sample to a batch of 96 samples that can be analyzed per run. The embodiments allow for both random access and batched testing, based on demands in the laboratory. The embodiments can be adapted for detecting microbes in other sample types such as fecal or skin samples, as well as microbes in food and environment samples. Long-read nucleic acid fragment sequence (LRS) data includes sequencing data of at least 1,000 base pairs or more of a DNA or an RNA molecule. The long-read nucleic acid fragment sequence data may be obtained using nanopore sequencing or PacBio sequencing or any other long-read sequencing technique.

Predefined abundance level comprises a level of abundance considered statistically significant from the perspective of identification of a microorganism in a sample. The predefined abundance level may include a level wherein the total number of bases sequenced in a sample is equal to or greater than the genome size of a given species.

Reference genomes include genomes corresponding to a variety of species that may be potentially present in a sample. Reference genomes may be stored in a genome database populated by routine clinical analysis of samples by total nucleic acid extraction and long-read ligation. A subset of reference genomes are selected based on the taxonomic identifiers assigned to LRS data obtained from a sample. The selection of a subset of reference genomes advantageously avoids the need for alignment of a large volume of LRS data with a large number of reference genomes making the methods of the embodiments computationally feasible. The subset of reference genomes may also be referred to as representative genomes or candidate reference genomes.

Aligning the LRS data with the candidate genomes includes matching nucleotides of the LRS data with the genome. Alignment can be measured or quantified by an identity score that may be defined as

$$\frac{total number of matched nucleotides}{alignment length}$$

or a read-coverage score defined as

$$\frac{alignment length}{read length} .$$

Coverage analysis comprises the calculation of the percentage of the breadth of coverage for each candidate genome based on the alignment results. The outcome of the coverage analysis may be represented in the form of a coverage distribution graph as illustrated in FIG. 2.

Some embodiments relate to a method for treating infection by one or more microorganism in a subject, the method comprising: determining long-read nucleic acid fragment sequence data (LRS data) from a sample obtained from the subject; performing taxonomic classification of the LRS data to assign one or more taxonomic identifiers to each record on the LRS data; determining abundance levels of a plurality of reference genomes based on the taxonomic identifiers of the LRS data; aligning the LRS data with the candidate genomes in response to one or more candidate genomes of the plurality of reference genomes reaching or exceeding a predefined abundance level; performing coverage analysis based on the alignment of the LRS data with the candidate genomes; determining an identity of one or more microorganism present in the sample based on the coverage analysis so as to detect infection by the one or more microorganism in the subject; and administering a therapeutic agent to the subject when infection by the one or more microorganism is detected in the subject.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A clinical decision support system comprising one or more processing units, the one or more processing units configured to:
receive long-read nucleic acid sequence data (LRS data) obtained from a sample, the LRS data comprising a plurality of records;
perform taxonomic classification of the LRS data to assign one or more taxonomic identifiers to each record on the LRS data;
determine abundance levels of a plurality of reference genomes in the sample based on the taxonomic identifiers;
align the LRS data with a subset of the reference genomes, wherein the subset of the reference genomes demonstrated abundance in the sample reaching or exceeding a predefined abundance level;
perform coverage analysis based on the alignment of the LRS data with the subset of the reference genomes to obtain a coverage estimate of each of the subset of the reference genomes; and
identify one or more microorganism species present in the sample based on the coverage estimate.

2-15. (canceled)

16. The system of claim 1, wherein the LRS data is obtained from culture-free clinical samples.

17. The system of claim 1, wherein each record of the LRS data comprises data of at least 1,000 base pairs, and
wherein the determination of the LRS data is performed in parallel with taxonomic classification.

18. The method of claim 1, wherein the determination of the LRS data, taxonomic classification and coverage analysis steps are performed in parallel.

19. The system of claim 1, wherein the coverage analysis is performed using a statistical distribution to estimate a breadth of coverage of the subset of the reference genomes by the LRS data.

20. The system of claim 19, wherein the statistical distribution is a Poisson distribution or a negative binomial distribution.

21. The system of claim 1, wherein the at least one processing unit is further configured to align records in the LRS data with records in an antimicrobial resistance genome database to determine presence of antimicrobial resistant species in the sample.

22. The system of claim 1, wherein performing taxonomic classification comprises determining a K-mer profile of each record in the LRS data.

23. The system of claim 22, wherein the K value is in the range of 3 to 31 nucleotides.

24. The system of claim 22, wherein assigning one or more taxonomic identifiers to each record in the LRS data is based on the K-mer profile of the respective records.

25. The system of claim 24, wherein the taxonomic identifiers represent an operational taxonomic unit (OTU) referring to one or a combination of one or more of: domain, kingdom, phylum, class, order, family, genus, species, strain, or individual genome.

26. The system of claim 25, wherein the subset of the reference genomes are selected based on the identified OTU.

27. The system of claim 1, wherein the aligning the LRS data to the subset of the reference genomes is based on a total number of matched nucleotides and a read coverage score.

28. The system of claim 1, wherein coverage analysis comprises determining a percentage of breadth of coverage of each genome in the subset of the reference genomes by the LRS data.

29. A computer-implemented method for microorganism identification, the method comprising:
receiving long-read nucleic acid fragment sequence data (LRS data) obtained from the sample;
performing taxonomic classification of the LRS data to assign one or more taxonomic identifiers to each record on the LRS data;
determining an abundance levels of a plurality of reference genomes based on the taxonomic identifiers of the LRS data;
aligning the LRS data with the subset of the reference genomes, wherein the subset of the reference genomes demonstrated abundance in the sample reaching or exceeding a predefined abundance level;
performing coverage analysis based on the alignment of the LRS data with the candidate genomes;
identifying one or more microorganism species present in the sample based on the coverage estimate.

30. A method for detecting infection by one or more microorganism in a subject, the method comprising:
determining long-read nucleic acid fragment sequence data (LRS data) from a sample obtained from the subject;
performing taxonomic classification of the LRS data to assign one or more taxonomic identifiers to each record on the LRS data;
determining abundance levels of a plurality of reference genomes based on the taxonomic identifiers of the LRS data;
aligning the LRS data with the candidate genomes in response to one or more candidate genomes of the plurality of reference genomes reaching or exceeding a predefined abundance level;
performing coverage analysis based on the alignment of the LRS data with the candidate genomes;
determining an identity of one or more microorganism present in the sample based on the coverage analysis so as to detect infection by the one or more microorganism in the subject.
